(19) United States
(12) Patent Application Publication
(10) Pub. No.: US 2011/0081401 A1
(43) Pub. Date: Apr. 7, 2011

(54) DERMAL PATCH COMPRISING CHONDRUS CRISPUS EXTRACT

(75) Inventors: Federico Panzieri, Agrate Brianza (MI) (IT); Giacomo Santus, Agrate Brianza (IT)

(73) Assignee: BIOPHIL ITALIA S.P.A., Agrate Brianza (Ml) (IT)

(21) Appl. No.: 12/936,452

(22) PCT Filed: Apr. 9, 2009
(86) PCT No.: PCT/EP09/02633

§ 371 Date: Dec. 6, 2010

(30) Foreign Application Priority Data
Apr. 10, 2008 (IT) MI2008A000636

(51) Int. Cl. A61K 9/70; A61K 38/00; A61K 31/715; B29C 39/38

(52) U.S. Cl. 424/443; 514/18.6; 514/54; 264/330

(57) ABSTRACT

The present invention relates to masks for dermal treatment or “patches”, based on a vegetal matrix, particularly red algae (Chondrus crispus) extract rich in polymeric thickeners. The invention relates also to compositions, in particular cosmetic and dermopharmaceutical formulations, to the process for their industrial preparation and to their use in cosmetic and pharmaceutical applications.

The present invention relates to a patch for dermal treatment.

More specifically, the subject of the invention is a mask for topical use (face mask), which is herein also referred to as “patch”, which allows the controlled release of active ingredients.

The dermal patch can be applied in all cases where the gradual and controlled release of the active ingredient in the epidermis is desirable. The different concentration of the active ingredient between the patch and the dermis induces a difference in the osmotic potential that produces a passage from the patch to the skin, overcoming the hydrophobic resistance typical of the skin.

Masks or patches for the skin treatment are well known in cosmetic technology, see for example EP-A-1603499.

Object of this invention is to provide a dermal patch which is biodegradable and of vegetal origin.

Another object of the invention is to provide a pre-shaped dermal treatment patch or mask that does not contain substances like TNT or polymers such as polyester or polyacrylate, in which the desired active ingredients can be inserted.

Another object of the invention is to provide a patch or mask that does not require an adhesive layer to be applied on the skin.

Yet another object is to provide a mask or patch that is free of preservatives and/or perfumes.

These objects—and others which will be described hereafter—are achieved by means of a dermal patch or mask containing a polysaccharide-based vegetal matrix, which is preferably extracted from red algae (Chondrus crispus) and which is rich in polymeric thickeners. Other aspects of the invention concern an industrial process for the preparation of the patch or mask and the use thereof in cosmetic and pharmaceutical applications.

The base of the patch or mask subject of this invention is a matrix of 100% plant origin, preferably obtained from an extract of red algae (Chondrus crispus) which contains polysaccharides with thickening properties and which have the peculiarity to form a biological network. Such polysaccharides are used in food and cosmetic products for their structuring properties, e.g. for enhancing viscosity and/or stability.

In the product subject of this invention, the polysaccharides of Chondrus crispus generate a network that imparts a three-dimensional structure to the product.

This structure is similar to that obtained e.g. by using silicones, and confer similar structural properties: transparency, elasticity and flexibility optimal to be easily applied on the skin; resistance, adhesive strength, possibility to be removed and replaced while maintaining the same shape, etc.

After use, the patch can be easily removed from the skin without leaving residues.

The patch or mask of this invention is a highly viscose fluid, structured in the form of a gel, which is resistant and can be safely applied to the human skin. The patch forms a film on the skin, allowing hydration by water release from its vegetal matrix as well as gradual and controlled release of any cosmetic, dermatological, or pharmaceutical active ingredient incorporated in the formulation.

These features are maintained as long as the water content of this structure remains unchanged: as the water evaporates, the structure tends to dry up and lose the above characteristics. However, the process of drying is much slower than with other cosmetic treatments of skin, usually from 10 to 30 min., occurring only several hours after air exposure.

Importantly, unlike the commonly used patches, which contain a synthetic polymeric support to guarantee the structure and mechanical characteristics of the patch, the matrix of the patch subject of this invention is totally natural and thus biodegradable.

The concentration of the ingredients in the composition is important for the patch effectiveness.

At lower concentrations of red algae (Chondrus crispus) extract, the required structural characteristics might not be obtained: the product forms a classical viscous gel, but remains liquid, and does not maintain the shape when subjected to mechanical stress, such as the removal from the blister or when applied on the skin.

Conversely, at higher concentrations of red algae extract, the mixture becomes clearer and fails to gel.

At specific concentrations, it is possible to obtain a gel structure with such interesting mechanical performance.

The stability of the dermal patch in its original sealed blister has been tested at 1, 3, and 12 months, by visual inspection and tests of adhesion to the skin, and no significant loss of property was observed.

Description of the Production Process

The compositions of the invention can be prepared by conveniently mixing the components of the mixture at suitable temperatures.

The blend of ingredients at the selected temperature is a viscose fluid that can be mixed with normal equipment, such as classical mixers.

The mixture is kept at constant temperature, from 30 to 90° C., preferably closer to the upper limit of 90° C., under constant stirring at 1 to 30 rpm, preferably around 10 rpm.

When cooled down gradually to room temperature, the obtained product is a highly resistant, transparent solid, showing high elasticity and adhesion to the skin. The appearance, texture and resistance of the finished product is comparable to those of similar silicone-based products, but with the advantage of being natural.

As already mentioned above, the product maintains these structural characteristics until the level of hydration is constant.

The product may contain any cosmetic or dermopharmaceutical active ingredient, incorporated in the solution or dispersed in the water entrapped by the network of polysaccharides.

The moulding takes place in a container—the “blister”—of the desired shape. As the liquid cools, the product takes the shape in the blister.

Accordingly, single patches of various size and shape can be prepared, suited to be used for different applications.

The exact amount of product can be dosaged through a timed and heated electronic valve under controlled temperature, specifically designed and adapted for this process. The correct opening time of the valve determines the flow of the product and the right dosage. The blister containing the product is then sealed, e.g. by an aluminium/polyethylene tie layer. Then the blister is die-cut to obtain the desired shape.

The sealed containers allow to retain the structural and mechanical properties of each patch until the moment of application.

In one embodiment of the invention, the initial mixture of ingredients is prepared by dispersing Chondrus Crispus (Phylcare® CC) in a blend of glycol, glycerin, or other moisturising substances until a homogeneous dispersion is obtained without any lumps.

In another embodiment, during the initial mixing phase, 75% of the water needed for the production of the batch is heated to 90° C. and after reaching this temperature, the suspension is added under vigorous stirring, and then it is left to cool to 50-60° C. In this embodiment, the water soluble active ingredient is dispersed in the remaining water, and then it is added to the hot phase under stirring to obtain a complete dispersion.

In a yet further embodiment, casting takes place preferably at a temperature between 50 and 60° C., and the dosage is performed through a nozzle connected to a valve attached to the interior of the mixer. The pneumatically controlled valve opens for the time necessary to let through the desired quantity of product, the opening time of the valve is adjusted by a pneumatic timer and the dosage is done in the same blister in which the product cools down, determining the product shape.

The entire process can be done in a sterile environment, using sterile materials, e.g. for the preparation of patches without preservatives, since the ingredients used in the mixture may provide a substrate for the proliferation of microorganisms (similar polysaccharides are used in the culture broths to incubate microorganisms in laboratory tests).

The process can also be carried out in an inert atmosphere of nitrogen, in order to limit any possible oxidative processes on the product.

Description of Use in Cosmetics and Dermopharmaceutical Applications.

The products obtained as described above can be used in both cosmetics and therapeutic applications.

For cosmetic/therapeutic uses, the products of the invention are appropriately formulated, possibly with other dermatologically active substances.

Formulations appropriate to the purpose of this invention include all types of cosmetic and dermopharmaceutical ingredients.

In addition to the ingredients used to formulate the products of this invention, the formulation may also contain other biologically active ingredients and excipients, such as surfactants, emollients, emulsifiers, solvents, moisturisers, enhancers of percutaneous absorption, and in general all types of ingredients, well-known to the formulators of cosmetic and pharmaceutical products.

Examples of mixtures used for different formulations and applications.

### EXAMPLE 1

### Base Formulation

| Ingredients | INCI Name | % |
|---|---|---|
| Water | Aqua | up to 100 |
| Methylpropanediol | Methylpropanediol | 10 |
| Glycerin | Glycerin | 20 |
| Chondrus crispus | Chondrus crispus | 2 |
| Active ingredient | INCI name of active | Proper quantity |
| | ingredient | |
| Preservative (if | Preservative | Proper quantity |
| desired) | systems | |

### EXAMPLE 2

### Base Formulation

| Ingredients | INCI Name | % |
|---|---|---|
| Water | Aqua | Up to 100 |
| Butylene glycol | Butylene glycol | 10 |
| Sorbitol solution 70% | Sorbitol, Aqua | 20 |
| Chondrus crispus | Chondrus crispus | 2 |
| Active ingredient | INCI name of active | Proper quantiy |
| | ingredient | |
| Preservative (if | Preservative | Proper quantity |
| desired) | systems | |

Examples of Use of Patches as Carriers for Active Ingredients.

Different patches have been prepared incorporating active ingredients for cosmetic use.

One example of patch uses Phylcare® Lyoserin, an high-molecular weight sericin, as described in Table 1.

TABLE 1

| Ingredients | INCI Name | % |
|---|---|---|
| Water | Aqua | 67 |
| Methylpropanediol | Methylpropanediol | 10 |
| Glycerin | Glycerin | 20 |
| Phylcare ® CC | Chondrus crispus | 2 |
| Phylcare ® Lyoserin | Sericin | 1 |
| KD 500 | | |

The method of preparation includes the following steps:

1. Mix all components, adding the components in the order shown above, heating at a temperature above 70° C., preferably at 90° C., under stirring.

2. Cast the mixture in the blisters, keeping the temperature of the nozzles preferably at around 50-60° C.

3. Close/seal the blister and allow to cool down to room temperature.

More specifically, the production of the mask takes place in a planetary mixer, equipped with a jacketing for heating with temperature control (thermostat).

The preparation is conducted as follows:

The product Phylcare® CC is pre-dispersed in the mixture of glycerin and methylpropanediol until it forms an homogeneous dispersion without lumps. In the meantime, 75% of the water needed for the production of the batch is heated to 90° C.

After reaching this temperature, the suspension is added to the water under vigorous stirring, then it is left to cool to 50-60° C.

The water soluble active ingredient is dispersed in the remainder of the water, and then added to the hot phase under stirring, to obtain a complete dispersion. Finally the casting of the product can be started.

The casting takes place preferably at the temperature of 50 and 60° C., using a dosage system.

The dosage is performed by a nozzle connected to a valve, attached to the interior of the mixer; the pneumatically controlled valve opens for the time necessary to let through the desired quantity of product. The opening time of the valve is adjusted by a pneumatic timer.

The dosage is done in the same blister which, with the cooling, imparts the shape to the product.

The product is then allowed to cool down, and an aluminium/polythene tie layer sheet is applied to seal the blister.

The blister is then die-cut, reaching the desired final shape of the finished product.

The sericin used in this formulation is one of the two families of proteins that constitute raw silk from the gland of the silkworm, its size ranging from 60 to 500 kDa.

Recently, several interesting properties of sericin were reported in the scientific literature regarding its affinity for the skin and human hair, as well as the high adherence to primary culture fibroblasts of human skin.

Phylcare® Lyoserin KD 500 is a sericin of high molecular weight, with very interesting properties in the field of cosmetic applications. It is an ingredient that helps to diminish the effects of inflammation.

In particular, Phylcare® Lyoserin KD 500 reduces the release of the inflammation mediator Interleukin-1α. This activity has been tested by means of in vitro tests.

Phylcare® Lyoserin KD 500, in addition, helps the protection of the skin against aggressive agents: this was demonstrated by means of in vitro test on cellular vitality after exposure to SDS (sodium dodecyl sulphate).

The ingredient has been dispersed in significant amount in the base mixture of the masks, during the final step of preparation.

The efficacy of a patch with this formulation for the eyes contour area has been clinically tested on volunteers to evaluate the hydrating, emollient and protective efficacy.

The test has been carried out on 20 female volunteers with sensitive skin, in the contour area which needs protection, hydration and decongestion.

Masks have been applied for 20 minutes on dry and clean skin, on both sides of the face, and after removal no other products have been applied.

The clinical and instrumental evaluations have been performed before the application of the products, and then after 7 days, 15 days, and 30 days of continuous daily application of the products. The results were completely positive.

The immediate effect after the first application of the product has been also evaluated. In this case the product has been applied only on one part of the face, while the other part has been used for control. The clinical and instrumental evaluations have been performed before the application of the product, and after 30 minutes from its removal.

The clinical evaluations included swelling and reddening of the eyelids.

The instrumental evaluations included the grade of hydration, TEWL (Trans Epidermal Water loss), and the erythema index.

Tests have shown that the application of the mask with Phylcare® Lyoserin KD 500 significantly reduces the erythema index in the monitored skin area—which is an indication of pre-inflammatory situations—and the liquids presence typical of eye bags effect. In addition, the product is able to positively affect skin hydration, keeping an optimal hydration index, and to protect the skin from dehydration by reducing the trans epidermal water loss.

A second examplary patch contains sodium hyaluronate, as described in table 2.

TABLE 2

| Components | INCI Name | % |
|---|---|---|
| Water | Aqua | 67.5 |
| Metylpropanediol | Methylpropanediol | 10 |
| Glycerin | Glycerin | 20 |
| Phylcare ® CC | Chondrus crispus | 2 |
| Phylcare ® Sodium | Sodium Hyaluronate | 0.3 |
| Hyaluronate XS | (<90 kDa) | |
| Phylcare ® Sodium | Sodium Hyaluronate | 0.2 |
| hyaluronate MW | (1000-1800 kDa) | |

The method of preparation is similar to that of the previous example.

This patch has been shown to recover the correct hydration level and is useful to treat the most dry/dehydrated areas of the skin.

The efficacy of this eye contour patch has been tested successfully in clinical studies on volunteers, where the hydrating and decongesting efficacy for this area has been evaluated.

The test was carried out with a method similar to that used in the previous example, which allowed to establish that the application of the mask with sodium hyauronate significantly improves the rehydration of dry skin, protects the stratum corneum reducing the trans epidermal water loss and helps to keep an optimal hydration index.

In addition, the regular application of the product smooths the skin in the eyes contour area, reducing also the evidence of slight wrinkles.

Therefore, the invention products fulfil the requirements for dermal masks, preferentially for face treatment, by delivering hydrosoluble active ingredients in a time-controlled manner on the human skin, by means of osmotic processes and occlusive effect.

The higher concentration of active ingredient present in the patch, compared to the derma, induces a difference of osmotic potential, which generates a flow from the patch to the skin, reducing in this way the resistance due to the hydrophobic character of the epidermis.

There are several advantages associated with the present invention respect to the common patches used for cosmetic and dermopharmaceutical applications, for example:
the matrix is completely vegetal and biodegradable;
the products show optimal structural features: elasticity, flexibility, resistance, adhesiveness, possibility to be removed and re-applied keeping the original shape, transparency;
there is no need for pre-formed substrates like TNT or polymers such as polyester or polyacrylate, in which the desired active ingredients are inserted and which require also an adhesive layer for adhesion to the skin;
the patch itself has hydrating properties, thanks to its vegetable matrix structure;
it is possible to produce masks without preservatives and/or perfumes;
patches of any shape or size are easily obtained by casting the melted mixture into a proper mould (blister);
the mask as such has an hydrating and lenitive effect to the skin;
the masks are dermatologically safe.

1. Dermal patch containing a red algae polysaccharide-based matrix.

2. Dermal patch according to claim 1, wherein said red algae polysaccharide is an extract of Chondrus crispus.

3. Dermal patch according to claim 2, further containing methylpropanediol and glycerine as moisturising ingredients.

4. Dermal patch according to claim 2, further containing butylene glycol and sorbitol as moisturising ingredients.

5. Dermal patch according to claim 2, containing water, methylpropanediol, glycerin, Chondrus crispus, and optionally a preservative.

6. Dermal patch according to claim 5, containing (as weight %) 67% water, 10% methylpropanediol, 20% glycerin, 2% Chondrus crispus and optionally a preservative.

7. Dermal patch according to claim 2, containing water, butylene glycol, sorbitol, Chondrus crispus and optionally a preservative.

8. Dermal patch according to claim 7, containing 67% water, 10% butylene glycol, 20% sorbitol, 2% Chondrus crispus and optionally a preservative.

9. Dermal patch according to claim 2, further containing an active ingredient.

10. Dermal patch according to claim 9, wherein said active ingredient is sericin.

11. Dearmal patch according to claim 9, wherein said active ingredient is Sodium Hyaluronate.

12. Method for the preparation of a dermal patch according to claim 1, comprising the following steps:
i) provide a water mixture of the vegetable matrix, excipients and active ingredients;
ii) heat the mixture to a temperature between 30 and 90° C.;
iii) cast the mixture in plastic moulds keeping the temperature between 30 and 90° C.;
iv) cool the mixture to room temperature.

13. The method according to claim 12, wherein in step ii) the mixture is heated at 90° C. under mixing.

14. The method according to claim 12, wherein in step iii) the temperature is maintained at 50-60° C.

15. The method according to claim 12, wherein the mixing step i) is performed in a planetary mixer equipped with a jacketing for heating under controlled temperature.

16. The method according to claim 12, wherein in step iii) the mixture is casted in plastic moulds for blisters.

17. The method of claim 16, further comprising the application of a sheet of aluminium/polythene tie layer to seal the blister and the die-cut of the blister to obtain the desired final shape of the finished product.

18. Method of using the dermal patch according to claim 1, for cosmetic applications.

19. The Method of using a dermal patch according to claim 1 , for the preparation of a medicament.
